(19) United States
(12) Patent Application Publication
(10) Pub. No.: US 2022/0055920 A1
(43) Pub. Date: Feb. 24, 2022

(54) Method of Wastewater Treatment using Renewable Energy

(71) Applicant: Ali Fadlelmula Mohamed Ali, Hofuf (SA)

(72) Inventor: Ali Fadlelmula Mohamed Ali, Hofuf (SA)

(21) Appl. No.: 17/134,871

(22) Filed: Dec. 28, 2020

Related U.S. Application Data
(62) Division of application No. 16/996,053, filed on Aug. 18, 2020.

(51) Int. Cl. C02F 1/14; C12M 1/22; B01D 57/00; B01L 3/00; C02F 1/469

(52) U.S. Cl. CPC C02F 1/14; C12M 23/10; C02F 1/469; B01L 3/00; B01D 57/00

(57) ABSTRACT

Treatment of wastewater and particularly relates to a technique for wastewater treatment using renewable energy (RE) which is sole solar energy (SE). In bench scale experiment, a device is constructed to consist of two fixed upper and lower glass Petri dishes. Another device consists of plastic containers and both devices with the same volume of wastewater in the upper and distilled water in the lower container. The first device is placed on the bench beside window to be exposed to sunlight at room temperature and the other device is put outdoors under direct sunlight during daytime. A build-up of small circular water droplets starts to appear on the external bottom of upper container. Water droplets are allowed to fall freely in the lower container, pH of droplets water is about 7.1. Yield of freshwater is at a rate of approximately 300 ml freshwater from 400 ml wastewater per 48 hours.

### CROSS REFERENCE TO RELATED APPLICATION

This application is a Divisional application and claims the benefit of and takes priority from U.S. Utility application Ser. No. 16/996,053 filed on Aug. 18, 2020, the contents of which are herein incorporated by reference.

### BACKGROUND OF THE INVENTION

### Field of the Invention

The invention herein relates to a method of treatment of wastewater to produce freshwater using solar energy only.

### Description of the Related Art

Removal of clean water from wastewater, in the present invention, is based on studies of intracellular water which proposed existence of two forms of liquid water; not one form as is widely accepted. Water molecules are made up of one oxygen atom and two hydrogens. We utilized the fact of the existence of two forms in bulk water and used solar energy to extract water from wastewater. By infrared spectroscopy technique, hydrogen bonding strength in intracellular water was studied. The study revealed that water forms two structural types as there are only two distinct hydrogen bond strengths i.e. water exists as networks of molecules interconnected by hydrogen bonds (Wiggins and van Ryn, 1986). Theory of co-existence of micro-domains of different densities shows that, in one form, H atom lies on a straight line between two O atoms, keeping molecules apart and water less dense, in a structured form of low density water (LDW).

The other form, where H bonds are weak and bent allowing molecules to approach each other and increase density, is non-structured high density water (HDW). The important difference from classical osmotic theory is that in two-state water, pressure gradients displace the water equilibrium in either way. When the pressure is positive inducing HDW and/or when the pressure is negative inducing LDW.

It has been demonstrated that different water clusters contain binding energies that resemble the structural properties of the liquid and the liquid/vapor interface of water (Dang and Chang, 1997).

The most important consequence of the coexistence of the two micro-domains is that any solution in such water has an inbuilt reservoir of free energy that can be harnessed for assembly of solute molecules into ordered arrays. The source of the reservoir of energy was demonstrated experimentally that any solute which dissolves in water generates a pressure gradient, positive at the immediate surface of the solute and negative further out (Wiggins, 2009).

Distilled water has a neutral pH of 7. Water is an example of a neutral molecule made of one oxygen and two hydrogen atoms. A neutral molecule is considered stable, however, molecules are often in a state of flux. Molecules may break apart to form ions, which do have a positive or negative electrical charge. In this case, the likely combinations of ions would be positively charged hydrogen and oxygen bonded to hydrogen or hydroxide, resulting in a negative charge.

Both ions of a neutral salt must occupy the same type of micro-domain determined by the relative potencies of the individual ions in a form of chaotropes or kosmotropes. For example, CaSO₄ is sparingly soluble partly because both Ca²⁺ and SO₄² are potent kosmotropes, requiring a large displacement of the water equilibrium. CaCl₂, on the other hand is highly soluble, partly because Cl⁻ is a chaotrope, so that the displacement of the water equilibrium by Ca²⁺ is corrected by an opposite displacement by 2Cl⁻. The outcome is that both CaCl₂ and CaSO₄ partition into HDW but CaSO₄ much more strongly than CaCl₂ (Wiggins, 2008).

The biopolymers fixed charges at the cellular level, are always strong chaotropes (large univalent anionic or cationic groups), inducing HDW in the double layer. When the counter-ion is also a chaotrope the cumulative effect is such that water adjacent to the surface appears to become pure HDW and compensation for the pressure gradient consists predominantly in induction of LDW. To quote few examples: cations show a preference for LDW in the order: Cs⁺>K⁺>Na⁺>Li⁺>H⁺. The break occurs between K⁺ and Na⁺; i.e. K⁺ is a chaotrope and Na⁺ is a kosmotrope. Univalent anions show the same trends. Their order is I⁻>Br⁻>Cl⁻>F⁻. Here, the break is between Cl⁻ and F⁻. The only differences between the ions on either side of the break is size. In sodium chloride NaCl, Na⁺ is a kosmotrope and Cl⁻ is a chaotrope, so the displacement of water equilibrium by Na⁺ is corrected by opposite displacement by Cl⁻ leading to induction of LDW formation.

Hydrophobic molecules and small cations such as Na⁺, Li⁺, H⁺, Ca²⁺, Mg²⁺ have an affinity for HDW, it will attract some HDW to its surface, increasing the amount of HDW on the layer surface. On the other hand most anions has an affinity for LDW but cannot generate LDW immediately at the surface because that region is under positive pressure. It follows that water immediately adjacent to a surface is always HDW, irrespective of the particular properties of the surface, and towards the bottom LDW predominates.

The principal objective of wastewater treatment is generally to allow water to be disposed of without danger to human health, animal health or the environment. Using wastewater for Irrigation is an effective form of wastewater disposal as it is both disposal and utilization. The most appropriate wastewater treatment for effluent use in agriculture is that which will produce an effluent meeting the recommended quality guidelines both at low cost and simple technological requirements (Arar, 1988). It is desirable in developing countries to adopt a low level of treatment not only from the point of view of cost but also in acknowledgement of the difficulty of operating complex systems reliably. In many locations it will be better to design the system to accept a low-grade of effluent rather than an effluent which continuously meets a stringent quality standard. Nearly half a million deaths per year are attributed to inadequate drinking water supplies (Prüss-Ustün et al., 2019). Despite remarkable progress in extending access to improved water sources over the past decades, an estimated 2.2 billion people still rely on sources contaminated with faecal bacteria, the vast majority of whom live in rural areas of developing countries (UNICEF, 2019). As well, wastewater from animal production settings may be source of anti-microbial resistant bacteria in developing and developed countries like Germany which affect global Public Health (Savin et al., 2020).

Wastewater is collected from homes, businesses, and many industries, and delivered to plants for treatment. Treatment plants are usually build to clean wastewater for discharge into streams or ponds, or for reuse. Initially, the volume of clean water in the stream dilutes wastes. Then bacteria and microorganisms in the water consume the sewage and other organic matter, turning it into new bacterial cells, carbon dioxide and other products (Vesiland et al., 2002).

The world's production of industrial wastewater, according to a UNESCO report, will double by 2025. UNESCO identifies manufacturing as the most significant contributor of wastewater among all industrial activities (UNESCO, 2017).

The basic function of wastewater treatment is to speed up the natural processes by which water is purified. The treatment of wastewater comprises two basic stages, primary and secondary. In the primary stage, solids are settled down and removed. The secondary stage uses biological processes to further purify wastewater.

### Primary Treatment

The sewage flows through a screen to remove large floating objects such as rags and sticks that might clog pipes. Then it passes into a grit chamber, where sand and small stones settle to the bottom. A number of sedimentation tanks can be used to remove organic and inorganic matter along with other suspended solids by flowing sewage through them. If the speed is reduced the suspended solids will gradually sink to the bottom to form a mass of solids called sludge.

### Secondary Treatment

After effluent leaves the sedimentation tank in the primary stage it is pumped to a facility using trickling filter. The trickling filter is a bed of stones from three to six feet deep through which sewage passes to enable bacteria to multiply and consume most of the organic matter. Nowadays, the trend is to use activated sludge process instead of trickling filters. In this process, the sewage is pumped into an aeration tank, where it is mixed with air and sludge loaded with bacteria and allowed to remain for many hours to enable the bacteria to break down the harmful organic matter, bio-solids, into harmless by-products (Kinney et al., 2006). Then a disinfection phase follows by injecting sludge with chlorine to kill pathogenic bacteria and to reduce odour before being discharged into receiving waters. However, chlorine may be toxic to fish, so it further needs de-chlorination. Alternative to this, using ultraviolet light or ozone may be feasible as clean and safe. New pollution problems, such as heavy metals, chemical compounds, and toxic substances, have placed additional burdens on wastewater treatment systems. Burdens press heavily as demand for water reuse is ever increasing. A host of advanced waste treatment techniques has been developed ranging from biological treatment to physical-chemical separation techniques such as filtration, carbon adsorption, distillation, and reverse osmosis. It is worth mentioning that the more advanced and refined the techniques, the higher are the expenses.

Waste effluents purified by such techniques, can be used for industrial, agricultural, or recreational purposes, or even drinking water supplies (Watts, 1998). Conventional and innovative methods for wastewater treatment have drawback of the high consumption of energy. In addition, implication of these techniques in environmental issue is great by emission of greenhouse gases. Some of the reviewed methods above that utilize RE, used complicated technology to reach the final product. Therefore simple green methods to serve rural communities and industrial activities could prove to be breakthrough in light of availability of SE in different areas all over the World.

### SUMMARY OF THE INVENTION

1. The present invention relates to a method of treatment of wastewater in which domestic wastewater is treated in bench-scale experiments. Wastewater is used after primary treatment to remove large floating objects such as rags and sticks.

2. To achieve the above purpose, in one embodiment, glass Petri dishes consists of two plates of same size, arranged in a device as upper and lower plates. A volume of 60 ml of wastewater is put in the upper plate and 20 ml of distilled water in the lower plate.

3. The device arranged in step 2, is placed on the bench beside window to be exposed to sunlight at room temperature. After about 30 mins water droplets started to appear on the lower face of the upper plate. Water droplets increase in size with time and allowed to fall freely on the lower plate.

4. Volume of water droplets obtained in step 3 in lower plate was measured by the increase of water volume of the lower plate. After 48 hours, water in the upper plate dried out leaving waste debris and the volume of water in lower plate increased by approximately 44 ml i.e. the total volume is approximately 64 ml.

5. Further, in another bench scale experiment, arrange a device to consist of three glass Petri dishes and fix one over the other to make upper, middle and lower plates. Put 60 ml wastewater in each of the upper and middle plates and 20 ml of distilled water in the lower plate. Place the device beside a window to be exposed to sunlight. On examination the lower surface of the upper and middle plates after 30 minutes are cloudy and buildup of water droplets starts and is increased as time passes. Collect water droplets from the upper and middle plates with sterile glass rod to measure its volume. The water volume is approximately double the volume obtained in step 4.

In one embodiment, rounded plastic containers were used consist of two plates of same size arranged as upper and lower plates. A volume of 400 ml wastewater was put in the upper plate and 100 ml distilled water in lower plate. The device is put outdoors exposed to sunlight and was inspected regularly. After 48 hours, wastewater in upper plate dried out leaving solid waste debris and in the lower container freshwater was about 400 ml at experiment termination.

The droplet water obtained according to the present disclosure was at a rate of approximately 300 ml freshwater from 400 ml wastewater per 48 hours. pH of droplets water is about 7.1.

As compared to other methods of wastewater treatment, embodiments in the present disclosure have the following advantages:

1. Consumption of energy is low as it utilizes SE in all reactions till production of clean potable water.

2. It is simple and does not require complex infrastructure or sophisticated equipment.

3. The technique can be scaled up for large quantity of wastewater.

4. A green method with no greenhouse gases emission.

5. The problem of diurnal pattern of sunlight is solved as energy of infrared rays continues during night.

The details of one or more embodiments of the present invention are presented in the accompanying figures and the description below. Other features, objects, and advantages of the invention will be apparent from the description and figures and from the claims.

### BRIEF DESCRIPTION OF THE DRAWINGS

FIG. 1 illustrates bench scale experiment of two glass Petri dishes fixed one over the other with wastewater in the upper dish and distilled water in the lower dish. The device is exposed to sunlight according to embodiment 1.

FIG. 2 illustrates another bench scale experiment of three glass Petri dishes fixed one over the other and the device is exposed to sunlight according to embodiment 3.

FIG. 3 illustrates an outdoor experiment of two rounded plastic containers fixed one over the other and the device is placed outdoors to be exposed to direct sunlight according to embodiment 4.

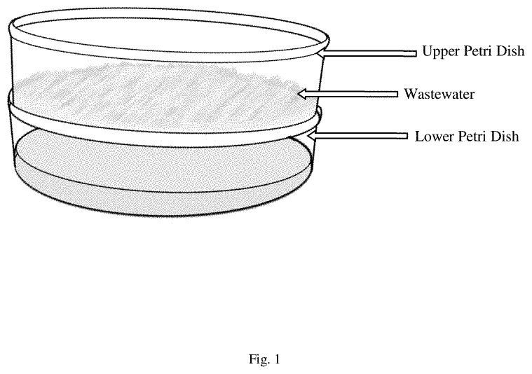

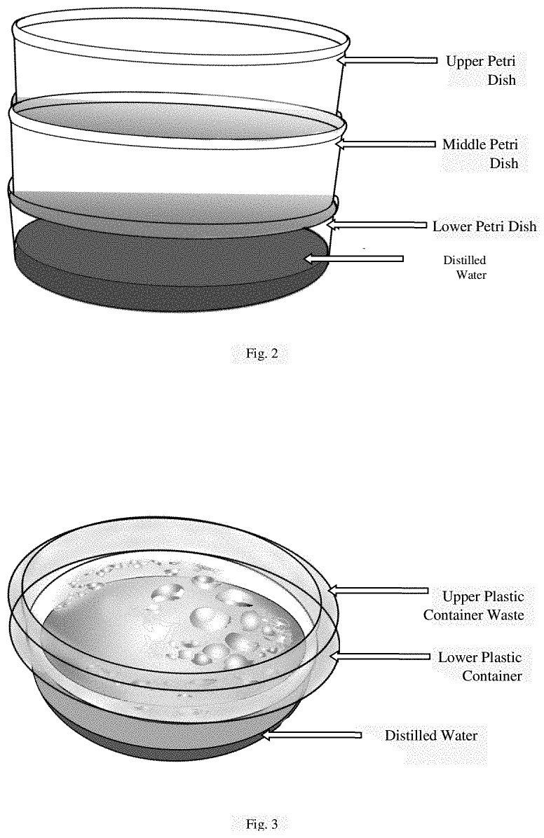

### DETAILED DESCRIPTION OF THE SEVERAL EMBODIMENTS

To make the present disclosure clear, the following embodiments give detailed description.

### Embodiment 1

In a bench scale experiment, a device consists of two glass Petri dishes, fixed one over the second to make upper and lower plates. A volume of 60 ml of wastewater is put in the upper plate and 20 ml of distilled water in the lower plate. Place the device beside a window to be exposed to sunlight. On examination the lower surface of the upper plate after 30 minutes is cloudy and buildup of water droplets starts and is increased as time passes. Water droplets increase in size with time and allowed to fall freely on the lower plate and was measured by the increase of water volume of the lower plate. After 48 hours, water in the upper plate dried out leaving waste debris and the volume of water in lower plate increased by approximately 44 ml.

### Embodiment 2

Further, in another bench scale experiment, arrange a device to consist of three glass Petri dishes and fix one over the other to make upper, middle and lower plates. Put 60 ml wastewater in each of the upper and middle plates and 20 ml of distilled water in the lower plate. Place the device beside a window to be exposed to sunlight. On examination the lower surface of the upper and middle plates after 30 minutes are cloudy and buildup of water droplets starts and is increased as time passes. Collect water droplets from the upper and middle plates with sterile glass rod to measure its volume. The water volume is approximately double the volume obtained in step 4.

### Embodiment 3

In an outdoor experiment, use rounded plastic containers of same size and arrange as upper and lower plates. A volume of 400 ml wastewater is put in the upper plate and 100 ml of distilled water in the lower plate. Place the device outdoors to be exposed to direct sunlight and was inspected regularly. After 48 hours, wastewater in upper plate dried out leaving debris while in the lower container freshwater was about 400 ml at experiment termination.

### Technical Description

In the present invention, we used SE in form of the entire electromagnetic spectrum that reach the earth surface as the sole source of energy. Making use of the fact that ordinary bulk water exists in two kinds: HDW and LDW prompted us to think of the present invention. For instance, in the upper container the positively-charged HDW lies in lower position and towards the container's upper side lies the negatively-charged LDW. The lower container contains distilled water with negatively-charged ions.

Hence, the negatively-charged distilled water ions in the lower container attracted the positively -charged HDW ions in the upper container through the bottom of the upper container that function as filter using the power of ‘unlike poles attract’. It provide fresh droplets water free of solute organic and inorganic matter in wastewater. Beside the power of “unlike poles attract” that induce emerging of circular water droplets through glass or plastic material in the bottom of the upper container, in the present invention, may be interpreted by the “quantum tunneling state” of the water. Discovery of this state of water contributes to the knowledge of utilization of energy by water. It has been reported that quantum tunneling allows particles to move through energy barriers and verified by using neutron scattering technology (Kolesnikov et al., 2016).

### REFERENCES

Dang, L. X. and Chang, T-M. (1997). Molecular dynamics study of water clusters, liquid, and liquid-vapor interface of water with many-body potentials. J. Chem. Phys. 106, 8149.

https://doi.org/10.1063/1.473820

Kinney, C. A., Furlong, E. T., Zaugg, S. D., Burkhardt, M. R., Werner, S. L., Cahill, J. D., and Jorgensen, G. R., 2006, Survey of organic wastewater contaminants in biosolids destined for land application: Environmental Science and Technology, v. 40, no. 23, p. 7207-7215.

doi:10.1021/es0603406.

Kolesnikov A. I., Reiter G. F., Choudhury N. et al. (2016). Quantum Tunneling of Water in Beryl: A New State of the Water Molecule. Phys. Rev. Lett. 116, 167802.

Prüss-Ustün A., Wolf J., Bartram J., Clasen T., Cumming O., Freeman M. C., Gordon B., Hunter P. R., Medlicott K., Johnston R. (2019). Burden of Disease from Inadequate Water, Sanitation and Hygiene for Selected Adverse Health Outcomes: An Updated Analysis with a Focus on Low- and Middle-Income Countries. Int. J. Hyg. Environ. Health. 2019;222:765-777. doi: 10.1016/j.ijheh.2019.05.004.

Savin, M. G. Bierbaum, J A. Hammerl, C. Heinemann, M. Parcina, E. Sib, A. Voigt, and J. Kreyenschmidt (2020).ESKAPE Bacteria and Extended-Spectrum-β-Lactamase-Producing Escherichia coli Isolated from Wastewater and Process Water from German Poultry Slaughterhouses.Appl Environ Microbiol. Apr; 86(8): e02748-19. doi: 10.1128/AEM.02748-19

UNESCO (2017).The United Nations world water development report, 2017: Wastewater: the untapped resource,

ISBN: 978-92-3-100201-4, 978-92-3-600072-5

Available from: WWW.UNESCO.ORG

UNICEF. WHO (2019). Progress on Household Drinking Water, Sanitation and Hygiene 2000-2017: Special Focus on Inequalities. World Health Organization; Geneva, Switzerland.

Vesiland, P. Aarne; Worrell, William; and Reinhart, Debra. (2002). Solid Waste Engineering. Australia: Brooks/Cole.

Watts, Richard J. (1998). Hazardous Wastes: Sources, Pathways, Receptors. New York: John Wiley & Sons.

Wiggins P (2008) Life Depends upon Two Kinds of Water. PLoS ONE 3(1): e1406. https://doi. org/10.1371/journal.pone.0001406

Wiggins, P. (2009). The Source of Some of the Extraordinary Powers and Properties of Enzymes. Water Journal.

doi: 10.14294.WATER.2009.1

Wiggins, P. M. and van Ryn, R. T. (1986). The solvent properties of water in desalination membranes. J. Macromol. Sci. Chem. A23: 875-903.

What is claimed is:

1. A method to detect the rate of extraction of freshwater from wastewater with time factor using a device for treatment of wastewater by fixing two glass Petri dishes as upper and lower plates. A volume of 60 ml wastewater, can be increased or decreased, is put in the upper plate and 60 ml distilled water, can be increased or decreased, in the lower plate. The device is exposed to sunlight but not to direct sunrays, at room temperature. The obtained water droplets which is collected from the lower surface of the upper dish. The obtained freshwater collected in the lower plate. Another device is arranged to consist of three glass Petri dishes and fix one over the other to make upper, middle and lower plates. A volume of 60 ml wastewater, can be increased or decreased, is placed in each of the upper and middle plates and 60 ml of distilled water, can be increased or decreased, in the lower plate. The device is exposed to sunlight but not to direct sunrays, at room temperature. The obtained water droplets which is collected, in small beakers, from the lower surface of the upper and middle plates, wherein a volume of 60 ml wastewater, can be increased or decreased, is put in the upper plate and 20 ml distilled water, can be increased or decreased, in the lower plate. The device is exposed to sunlight, but not to direct sunrays, at room temperature and observed for 48 hours. The obtained waste debris in the upper plate and droplets of water which is collected from the lower surface of the upper dish.
